SEQUENCE LISTING (9 sequences)

SEQ ID NO: 1 - DNA, 20 bp, Cricetulus griseus
  source 1..20: mol_type = genomic DNA
tacggtgtgacgtgcaccgg

SEQ ID NO: 2 - DNA, 20 bp, Cricetulus griseus
  source 1..20: mol_type = genomic DNA
gggagtcgaggatttgagta

SEQ ID NO: 3 - DNA, 20 bp, Cricetulus griseus
  source 1..20: mol_type = genomic DNA
aagttcagggcacgcgtggc

SEQ ID NO: 4 - DNA, 20 bp, Cricetulus griseus
  source 1..20: mol_type = genomic DNA
agcagggccccgttagacta

SEQ ID NO: 5 - DNA, 20 bp, Cricetulus griseus
  source 1..20: mol_type = genomic DNA
atcaccgcttgtccccctca

SEQ ID NO: 6 - DNA, 20 bp, Cricetulus griseus
  source 1..20: mol_type = genomic DNA
tctgcatctggctaaatggg

SEQ ID NO: 7 - DNA, 20 bp, Cricetulus griseus
  source 1..20: mol_type = genomic DNA
gagtcctcgaggccctagca

SEQ ID NO: 8 - DNA, 20 bp, Cricetulus griseus
  source 1..20: mol_type = genomic DNA
ctaatgagccaaccgggcag

SEQ ID NO: 9 - DNA, 20 bp, Cricetulus griseus
  source 1..20: mol_type = genomic DNA
ttcaagggctcagtgaccgt

(19) United States
(12) Patent Application Publication
(10) Pub. No.: US 2026/0337577 A1
(43) Pub. Date: Oct. 29, 2026

(54) CHINESE HAMSTER OVARY CELL GENOMIC HOTSPOTS FOR RECOMBINANT PROTEIN PRODUCTION

(71) Applicant: UNIVERSITY OF DELAWARE, Newark, DE (US)

(72) Inventors: Kelvin H. Lee, Newark, DE (US); William C. Hilliard, Amherst, NH (US)

(73) Assignee: University of Delaware, Newark, DE (US)

(21) Appl. No.: 19/115,713

(22) PCT Filed: Sep. 27, 2023
(86) PCT No.: PCT/US2023/075194

§ 371 Date: Mar. 26, 2025

Related U.S. Application Data
(60) Provisional application No. 63/410,358, filed on Sep. 27, 2022.

(51) Int. Cl. C12N 5/071; C07K 16/00; C12N 15/11; C12N 15/90

(52) U.S. Cl. CPC C12N 5/0682; C07K 16/00; C12N 15/11; C12N 15/90; C07K 2317/14; C12N 2510/02; C12N 2510/04

(57) ABSTRACT

The invention provides a recombinant Chinese Hamster Ovary (CHO) cell for producing a recombinant protein. The recombinant CHO cell comprises a heterologous gene encoding the recombinant protein, wherein the heterologous gene is integrated into the genome of the recombinant CHO cell at one or more integration sites, each of the one or more integration sites is within a predetermined distance, for example, 200 kb, from a target sequence in the genome, and the target sequence is a variant of, for example, at least 80% identical to, a gene sequence selected from the group consisting of Azin1, Sart1, Fosl2, Pde4a, Dop1b, Vgll4, Khdc4 and Ddc. Also provided are a method for producing the recombinant protein by the recombinant CHO cell and a method for preparing the recombinant CHO cell.

### CROSS-REFERENCE TO RELATED APPLICATION

This application claims priority to U.S. Provisional Application No. 63/410,358, filed Sep. 27, 2022, and the contents of which are incorporated herein by reference in their entireties for all purposes.

### REFERENCE TO U.S. GOVERNMENT SUPPORT

This invention was made with government support under NSF Grant No. 1736123 and NIST Grant No. 70NANB17H002 awarded by the National Science Foundation and National Institute of Standards and Technology, respectively. The government has certain rights in the invention.

### FIELD OF THE INVENTION

This invention relates generally to recombinant Chinese Hamster Ovary (CHO) cells for producing recombinant proteins.

### BACKGROUND OF THE INVENTION

Historically, CHO cell line development (CLD) for commercial bioprocesses involved the integration of transgenes encoding the therapeutic product into random genomic loci followed by copy number amplification, leading to a cell pool with significant genotypic and phenotypic heterogeneity. The rarity of stable high-producing cells within these pools necessitated the isolation and screening of hundreds or thousands of clones during a time- and resource-intensive procedure to identify a clonally-derived cell line with stable and acceptable productivity and product quality attributes, creating a significant bottleneck in the drug development pipeline. Targeted integration platforms, particularly those that employ recombinase-mediated cassette exchange (RMCE), can accelerate material generation for early product testing by offering unparalleled control over genotype and phenotype. However, the development of this type of CLD platform first requires knowledge of a precise location within the genome defined as a “hotspot” by its ability to facilitate stable and high transgene expression. A lack of pre-defined hotspots known to be capable of supporting multi-gram per liter mAb productivity therefore represents a significant barrier to widespread adoption of targeted integration for CHO CLD applications. Novel hotspots are typically found through random screening methods in which high-expressing single- or low-copy clones are isolated and genotyped. Although there have been some attempts to streamline screening protocols to find additional sites, all direct hotspot screening studies to date have been fundamentally limited in throughput by the requirement for single-cell cloning.

There remains a need for recombinant CHO cells with integrated transgenes at hot spots for stable and high expression.

### SUMMARY OF THE INVENTION

The inventors have surprisingly discovered a high-throughput screen that measures transgene transcription from precisely defined integration sites would provide a direct scoring metric, which could be used to prioritize sites for retargeting, in contrast to previous hotspot prediction methods relying on endogenous transcriptomic or epigenomic attributes.

A recombinant Chinese Hamster Ovary (CHO) cell for producing a recombinant protein is provided. The recombinant CHO cell comprises a heterologous gene encoding the recombinant protein, wherein the heterologous gene is integrated into the genome of the recombinant CHO cell at one or more integration sites, wherein each of the one or more integration sites is within 200 kb of a target sequence in the genome, and wherein the target sequence is at least 80% identical to a gene sequence selected from the group consisting of Azin1, Sart1, Fosl2, Pde4a, Dop1b, Vgll4, Khdc4 and Ddc.

The heterologous gene may be integrated into the genome of the recombinant CHO cell at the one or more integration sites in the one or more target sequences. The heterologous gene may be integrated into the genome of the recombinant CHO cell at the one or more integration sites within 200 kb of one of the one or more target sequences. The heterologous gene may be integrated into the genome of the recombinant CHO cell at the one or more integration sites in one of the one or more target sequences. The heterologous gene may be integrated into the genome of the recombinant CHO cell at one of the one or more integration sites in one of the one or more target sequences.

The recombinant CHO cell may be incubated in a culture medium and express the recombinant protein. The recombinant protein may be a monoclonal antibody.

For each recombinant CHO cell of the present invention, a method for producing a recombinant protein is provided. The production method may comprise incubating the recombinant CHO cell in a culture medium, and expressing the recombinant protein by the recombinant CHO cell. The production method may further comprise producing the recombinant protein at 0.1-10 g/L based on the total volume of the culture medium or 1-10 pg per said recombinant CHO cell per day. The production method may further comprise purifying the recombinant protein from the culture medium. The recombinant protein may be a monoclonal antibody.

A method for preparing a recombinant CHO cell is also provided. The preparation method comprises obtaining a host CHO cell comprising a genome having one or more target sequences, wherein each of the one or more target sequences is at least 80% identical to a gene sequence selected from the group consisting of Azin1, Sart1, Fosl2, Pde4a, Dop1b, Vgll4, Khdc4 and Ddc; and integrating a heterologous gene encoding a recombinant protein into the genome at one or more integration sites within 200 kb of the one or more target sequences, whereby a recombinant CHO cell is prepared.

The preparation method may further comprise integrating the heterologous gene into the genome at the one or more integration sites in the one or more target sequences.

The preparation method may further comprise integrating the heterologous gene into the genome at the one or more integration sites within 200 kb of one of the one or more target sequences.

The preparation method may further comprise integrating the heterologous gene into the genome at the one or more integration sites in one of the one or more target sequences.

The preparation method may further comprise integrating the heterologous gene into the genome at one of the one or more integration sites in one of the one or more target sequences.

According to the preparation method, the genome may comprise a landing pad.

The preparation method may further comprise incubating the recombinant CHO cell in a culture medium, and expressing the recombinant protein. The preparation method may further comprise purifying the recombinant protein from the culture medium.

According to the preparation method, the recombinant protein may be a monoclonal antibody.

### BRIEF DESCRIPTION OF THE DRAWINGS

FIGS. 1A-F show a Thousands of Reporters Integrated in Parallel (TRIP) system summary. A) Pooled plasmid library cloning by restriction digestion/ligation (only the Piggybac-compatible plasmid is shown). B) Establishment of TRIP pools derived from a CHO-K1 host by lentivirus transduction or Piggybac transposase-mediated integration. C) Preparation of NGS libraries for quantification of barcode mRNA expression and DNA copy number by targeted amplicon sequencing and integration site mapping by inverse PCR. D) Normalized expression of lentivirus and Piggybac TRIP barcodes as a function of distance between the barcode integration site and the nearest genomic CpG island. E) Expression distributions of barcodes grouped by A/B compartmentalization defined by Hi-C data described previously by Hilliard and Lee (Biotechnol. Bioeng. 118, 659-675 (2021)) for this CHO-K1 host. *=p<10⁻²³⁰, effect size=0.330 (Wilcoxon rank sum test). F) Distributions of barcode expression grouped by integration sites overlapping with ChIP-seq signal peak regions. Groups “H3K27ac”, “H3K4me3”, and “H3K4me1” refer to integration sites that overlap regions with significant signal peaks for each of those markers in the absence of any other modification. *=p<0.01 and moderate or large effect size (Wilcoxon rank sum test). ITR=Inverted terminal repeat, pA=polyadenylation signal.

FIG. 2 shows mRNA expression of the CD4-FKBP transgene in the landing pad in sorted polyclonal pools and clonally-derived master host cell lines with verified on-target, single-copy integrations at eight Piggybac TRIP integration sites and the Fer1l4 integration site. Absolute quantification of CD4-FKBP and RAB10 mRNA was performed by multiplexed ddPCR analysis. CD4-FKBP mRNA expression was driven by a CMV promoter in the landing pad and therefore considered to be representative of transgene expression at each site in the master host cell lines. RAB10 was chosen as a housekeeping gene based on its stable expression across several CHO host cell lines in an internal meta-analysis of public CHO RNAseq data (data not shown). Error bars represent Poisson 95% confidence intervals from three technical replicates.

FIG. 3A-B show a summary of RMCE configuration optimization experiments with GFP-Puro^{r}. A) RMCE was performed at three sites in clonally-derived cell lines, each with a verified on-target, single-copy landing pad integration, to integrate an RMCE cargo with various configurations. B) Plasmid configuration- and site-dependent GFP-Puro^{r} mRNA and protein expression. Error bars represent the standard deviation of median GFP-Puro^{r} fluorescence levels and mRNA expression levels of three biological replicate stable pools measured by flow cytometry and ddPCR, ignoring technical error. Ins=Insulator, CpG=Azin1 CpG island, ITR=Piggybac inverted terminal repeat, SV40L=SV40 late polyadenylation signal.

FIGS. 4A-D show a summary of mAb RMCE configuration optimization experiments with NISTmAb and Trastuzumab. A) Twelve combinations of upstream regulatory elements, internal regulatory elements and light chain/heavy chain orientations were tested for each mAb in RMCE cargo plasmids. Plasmid component abbreviations: NLC=NISTmAb light chain, NHC=NISTmAb heavy chain, TLC=trastuzumab light chain, THC=trastuzumab heavy chain, CpG=Azin1 CpG island, 5′ ITR=Piggybac 5′ inverted terminal repeat, SV40L=SV40 late polyadenylation signal. B) 4-day batch screen results. A two-point estimate was used to calculate qP (Day 0 and Day 4). C) Cell growth profile during fed-batch analysis of top candidate RMCE donor plasmids chosen based on the batch screen. D) mAb productivity over the course of the fed-batch experiment. Error bars indicate the standard deviation of three biological replicate pools, ignoring technical error.

### DETAILED DESCRIPTION OF THE INVENTION

The present invention relates to a recombinant Chinese Hamster Ovary (CHO) cell for producing a recombinant protein, in which a heterologous gene encoding a recombinant protein is integrated into the genome of the recombinant CHO cell at specific integration sites, also referred to as “hot spots,” for improving production of the recombinant protein by the CHO cell. The invention is based on the discovery by the inventors of a compendium of thousands of candidate sites identified with a high-throughput screen method that would likely capture the entire range of position-dependent expression that is possible for a single-copy transgene driven by a particular promoter, and that one high-throughput screening experiment could therefore effectively reduce the hunt for the highest-expressing stable hotspots to the practice of retargeting a handful of highly ranked sites and evaluating their ability to express a product of interest in the CHO sublineage and culture conditions used in any particular lab.

The inventors report the adaptation of Thousands of Reporters Integrated in Parallel (TRIP), a pooled high-throughput screening method capable of simultaneous measurement of mRNA expression, DNA copy number, and mapping information of barcoded transgenes integrated at thousands of genomic loci, for the application of identifying stable hotspots in the CHO genome. Genome-wide position effects on CMV promoter-driven transgene expression strength and stability were evaluated using barcoded reporters compatible with Piggybac transposase-mediated and lentiviral integration systems, which both integrate their cargo with well-known preferences for transcriptionally permissive chromatin regions and have been used previously for either CHO CLD or hotspot screening. Eight integration sites covering the top 50% of the transgene expression range measured by the Piggybac TRIP system were retargeted for integration of a RMCE-capable landing pad construct by CRISPR-mediated homology-directed repair (HDR) to assess reproducibility and clonal variability in a context directly translatable to an industrial CHO CLD platform. Stable integrant pools and clonally-derived cell lines with landing pad integrations at these sites exhibited equivalent or higher transgene mRNA expression than the industrially viable hotspot in the Fer1l4 locus in equivalent culture conditions, demonstrating the potential of these sites for commercial use.

The terms “protein” and “polypeptide” are used herein interchangeably, and refer to a polymer of amino acid residues with no limitation with respect to the minimum length of the polymer. Preferably, the protein or polypeptide has at least 20 amino acids. The definition includes both full-length proteins and fragments thereof, as well as modifications thereof (e.g., glycosylation, phosphorylation, deletions, additions and substitutions).

The term “polynucleotide” used herein refers to a polymer of nucleotide residues with no limitation with respect to the minimum length of the polymer. Preferably, the polynucleotide has at least 60 nucleotides. The polynucleotide may be a DNA, cDNA or RNA. A polynucleotide is native to a cell where the polynucleotide is naturally occurring in the cell. A polynucleotide is heterologous to a cell where the polynucleotide is not naturally occurring in the cell.

The term “variant” of a protein or polynucleotide used herein refers to a polypeptide having an amino acid or a polynucleotide having a nucleic acid sequence that is the same as the amino acid or nucleic acid sequence of a target protein or polynucleotide except having one or more amino acids or nucleic acids modified, for example, deleted, inserted, or replaced, respectively. A variant of a protein or polynucleotide may have an amino acid or nucleic acid sequence at least about 70%, 80%, 90%, 95%, or 99%, preferably at least about 80%, more preferably at least about 90%, identical to the amino acid sequence or nucleic acid of the protein or polynucleotide.

The term “host CHO cell” as used herein refers to a CHO cell before a heterologous gene is integrated into its genome at one or more integration sites within a predetermined distance from a target sequence in the genome, which target sequence is a variant to a gene sequence selected from the group consisting of Azin1, Sart1, Fosl2, Pde4a, Dop1b, Vgll4, Khdc4 and Ddc.

The present invention provides a recombinant Chinese Hamster Ovary (CHO) cell for producing a recombinant protein. The CHO cell comprises a heterologous gene encoding the recombinant protein. The heterologous gene is integrated into the genome of the recombinant CHO cell at one or more integration sites. Each integration site is within a predetermined distance from a target sequence in the genome. The target sequence is a variant to a gene sequence selected from the group consisting of Azin1, Sart1, Fosl2, Pde4a, Dop1b, Vgll4, Khdc4 and Ddc. Table 1 shows the mapping information of Azin1, Sart1, Fosl2, Pde4a, Dop1b, Vgll4, Khdc4 and Ddc.

The heterologous gene encoding the recombinant protein is a DNA not naturally occurring in a CHO cell. The heterologous gene may be integrated into the genome of the recombinant CHO cell at one or more integration sites by any method.

Each integration site may be within 200 kb, 100 kb, 50 kb, 25 kb, 10 kb, 5 kb, 1 kb, 500 bases, 100 bases, 50 bases, 10 bases, 5 bases or 1 base of the target sequence in the genome. The target sequence may be at least about 70%, 80%, 90%, 95%, or 99%, preferably at least about 80%, more preferably at least about 90%, Identical to a gene sequence, which may be Azin1, Sart1, Fosl2, Pde4a, Dop1b, Vgll4, Khdc4 or Ddc.

In one embodiment, the heterologous gene may be integrated into the genome of the recombinant CHO cell at one or more integration sites in one or more target sequences in the genome, where each target sequence is at least about 70%, 80%, 90%, 95%, or 99%, preferably at least about 80%, more preferably at least about 90%, identical to Azin1, Sart1, Fosl2, Pde4a, Dop1b, Vgll4, Khdc4 or Ddc.

In another embodiment, the heterologous gene may be integrated into the genome of the recombinant CHO cell at one or more integration sites, where each integration site is within 200 kb, 100 kb, 50 kb, 25 kb, 10 kb, 5 kb, 1 kb, 500 bases, 100 bases, 50 bases, 10 bases, 5 bases or 1 base of one target sequence in the genome, and where the target sequence is at least about 70%, 80%, 90%, 95%, or 99%, preferably at least about 80%, more preferably at least about 90%, identical to Azin1, Sart1, Fosl2, Pde4a, Dop1b, Vgll4, Khdc4 or Ddc.

In yet another embodiment, the heterologous gene may be integrated into the genome of the recombinant CHO cell at one or more integration sites in one target sequence, where the target sequence is at least about 70%, 80%, 90%, 95%, or 99%, preferably at least about 80%, more preferably at least about 90%, identical to Azin1, Sart1, Fosl2, Pde4a, Dop1b, Vgll4, Khdc4 or Ddc.

In yet another embodiment, the heterologous gene may be integrated into the genome of the recombinant CHO cell at one integration site in one target sequence, where the target sequence is at least about 70%, 80%, 90%, 95%, or 99%, preferably at least about 80%, more preferably at least about 90%, identical to Azin1, Sart1, Fosl2, Pde4a, Dop1b, Vgll4, Khdc4 or Ddc.

The recombinant protein may be any protein. Although the recombinant protein may be a protein naturally expressed in a natural CHO cell, the recombinant CHO cell may overexpress the protein as compared with the natural CHO cell. The recombinant protein may be heterologous to a natural CHO cell. The protein may be an antibody, for example, a monoclonal protein. The recombinant CHO cell may be incubated in a culture medium and expresses the recombinant protein.

For each recombinant CHO cell of the present invention, a method for producing the recombinant protein is provided. The production method comprises incubating the recombinant CHO cell of the present invention in a culture medium, and expressing the recombinant protein by the recombinant CHO cell.

The production method may further comprise producing the recombinant protein at 0.1-10, 0.5-10, 1-10, 5-10, 0.1-9, 0.5-9, 1-9, 5-9, 0.1-8, 0.5-8, 1-8, 5-8, 0.1-7, 0.5-7, 1-7, 5-8, 0.1-7, 0.5-7, 1-7, 5-7, 0.1-6, 0.5-6, 1-6, 5-6, 0.1-5, 0.5-5, 1-5, 0.1-1 or 0.1-0.5 g/L, based on the total volume of the culture medium, or 1-10, 1-9, 1-8, 1-7, 1-6, 1-5, 1-4, 1-3 or 1-2 pg per said recombinant CHO cell per day.

The production method may further comprise purifying the recombinant protein from the culture medium. The purification of the recombinant protein may be achieved by any method.

According to the production method, the recombinant protein may be any protein. The protein may be an antibody, for example, a monoclonal protein. The recombinant CHO cell may be incubated in a culture medium and expresses the recombinant protein.

A method for preparing a recombinant CHO cell is provided. The preparation method comprises obtaining a host CHO cell. The host CHO cell comprises a genome having one or more target sequences. Each target sequence is a variant of a gene sequence selected from the group consisting of Azin1, Sart1, Fosl2, Pde4a, Dop1b, Vgll4, Khdc4 and Ddc. The preparation further comprises integrating a heterologous gene encoding a recombinant protein into the genome at one or more integration sites. Each integration site is within a predetermined distance from a target sequence in the genome. The target sequence is a variant to a gene sequence selected from the group consisting of Azin1, Sart1, Fosl2, Pde4a, Dop1b, Vgll4, Khdc4 and Ddc. As a result, the recombinant cell is prepared.

According to the preparation method, the heterologous gene encoding the recombinant protein is a DNA not naturally occurring in a CHO cell. The heterologous gene may be integrated into the genome of the recombinant CHO cell at one or more integration sites by any method.

According to the preparation method, each integration site may be within 200 kb, 100 kb, 50 kb, 25 kb, 10 kb, 5 kb, 1 kb, 500 bases, 100 bases, 50 bases, 10 bases, 5 bases or 1 base of the target sequence in the genome. The target sequence may be at least about 70%, 80%, 90%, 95%, or 99%, preferably at least about 80%, more preferably at least about 90%, identical to a gene sequence, which may be Azin1, Sart1, Fosl2, Pde4a, Dop1b, Vgll4, Khdc4 or Ddc.

In one embodiment, the preparation method comprises integrating the heterologous gene into the genome of the recombinant CHO cell at one or more integration sites in one or more target sequences in the genome, where each target sequence is at least about 70%, 80%, 90%, 95%, or 99%, preferably at least about 80%, more preferably at least about 90%, identical to.

In another embodiment, the preparation method comprises integrating the heterologous gene into the genome of the recombinant CHO cell at one or more integration sites, where each integration site is within 200 kb, 100 kb, 50 kb, 25 kb, 10 kb, 5 kb, 1 kb, 500 bases, 100 bases, 50 bases, 10 bases, 5 bases or 1 base of one target sequence in the genome, and where the target sequence is at least about 70%, 80%, 90%, 95%, or 99%, preferably at least about 80%, more preferably at least about 90%, identical to Azin1, Sart1, Fosl2, Pde4a, Dop1b, Vgll4, Khdc4 or Ddc.

In yet another embodiment, the preparation method comprises integrating the heterologous gene into the genome of the recombinant CHO cell at one or more integration sites in one target sequence, where the target sequence is at least about 70%, 80%, 90%, 95%, or 99%, preferably at least about 80%, more preferably at least about 90%, identical to Azin1, Sart1, Fosl2, Pde4a, Dop1b, Vgll4, Khdc4 or Ddc.

In yet another embodiment, the preparation method comprises integrating the heterologous gene into the genome of the recombinant CHO cell at one integration site in one target sequence, where the target sequence is at least about 70%, 80%, 90%, 95%, or 99%, preferably at least about 80%, more preferably at least about 90%, identical to Azin1, Sart1, Fosl2, Pde4a, Dop1b, Vgll4, Khdc4 or Ddc.

According to the preparation method, the genome of the host CHO cell may comprise a landing pad. The preparation method may further comprise swapping a partial or full sequence of the landing pad with the heterologous gene in the integration step such that the genome of the prepared recombinant CHO cell comprises the integrated heterologous gene and excludes the partial or full sequence of the landing pad.

The preparation method may further comprise incubating the recombinant CHO cell in a culture medium, and expressing the recombinant protein. The recombinant protein may be produced by the recombinant CHO cell at 0.1-10, 0.5-10, 1-10, 5-10, 0.1-9, 0.5-9, 1-9, 5-9, 0.1-8, 0.5-8, 1-8, 5-8, 0.1-7, 0.5-7, 1-7, 5-8, 0.1-7, 0.5-7, 1-7, 5-7, 0.1-6, 0.5-6, 1-6, 5-6, 0.1-5, 0.5-5, 1-5, 0.1-1 or 0.1-0.5 g/L, based on the total volume of the culture medium, or 1-10, 1-9, 1-8, 1-7, 1-6, 1-5, 1-4, 1-3 or 1-2 pg per said recombinant CHO cell per day.

The preparation method may further comprise purifying the recombinant protein from the culture medium. The purification of the recombinant protein may be achieved by any method.

According to the preparation method, the recombinant protein may be any protein. The protein may be an antibody, for example, a monoclonal protein.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### Example 1. A Compendium of Stable Hotspots in the CHO Genome

The use of targeted integration for industrial CHO cell line development currently requires significant upfront effort to identify genomic loci capable of supporting multi-gram per liter therapeutic protein production from a limited number of transgene copies. To address this barrier to widespread adoption, the inventors characterized transgene expression from thousands of stable hotspots in the CHO genome using the Thousands of Reporters Integrated in Parallel high-throughput screening method. This genome-scale dataset was used to define a limited set of epigenetic properties of hotspot regions with sizes on the order of 10 kb. Cell lines with landing pad integrations at eight retargeted hotspot candidates consistently exhibited higher transgene mRNA expression than a commercially viable hotspot in equivalent culture conditions. Initial benchmarking of NISTmAb and trastuzumab productivity from one of these hotspots yielded mAb productivities of approximately 0.7 to 2 g/L (specific productivity, qP, range: 2.9-8.2 pg/cell/day) in small-scale fed-batches. These findings indicate the list of hotspot candidates identified by the inventors are a valuable resource for targeted integration platform development within the CHO community.

### A. Materials and Methods

### 1. Molecular Cloning

The Piggybac (pPB-GFP) and lentiviral (pLV-GFP) base, non-barcoded cargo plasmids were assembled using standard molecular cloning techniques. Additionally, the tamoxifen-inducible Piggybac transposase described by Akhtar et al. (Nat. Protoc. 9, 1255-1281(2014)) with minor modifications to replace rarely used codons in CHO was inserted into an in-house plasmid backbone to generate pPBase-IRES-mCherry. Barcoded plasmid pools (pPB-GFP-bc and pLV-GFP-bc) were generated by restriction digestion/ligation cloning followed by high-efficiency transformations with Endura Electrocompetent E. coli (Lucigen) using a GenePulser Xcell (BioRad). Bxb1-compatible RMCE cargo plasmids were derived from a custom Golden Gate destination vector. Entry vectors were cloned by insertion of interchangeable plasmid parts (i.e. CMV promoters, transgene coding sequences, polyA signals, other regulatory elements) flanked by PaqCI recognition sequences into pCR™-Blunt II-TOPO™ using a Zero Blunt™ TOPO™ PCR Cloning Kit (Thermo Fisher). Assembly reactions with the destination and entry vectors were performed following the standard thermocycling protocol described by New England Biolabs for Golden Gate assembly.

### 2. Lentivirus Packaging

The pLV-GFP-bc plasmid library was packaged into lentiviruses by transient transfection of adherent HEK293LTV cells (Cell BioLabs) with Lipofectamine™ 3000 (Thermo Fisher) in a vented T75 flask (Corning) following a protocol from Thermo Fisher for lentiviral production modified to replace ViraPower packaging plasmid mix and lentiviral vector with an equal total mass of pMDLg/pRRE (Addgene plasmid #12251), pRSV-Rev (Addgene plasmid #12253), pMD2.G (Addgene plasmid #12259). Harvested, filtered supernatant was aliquoted into cryovials without concentrating and stored at −80 °C. Infectious titer was quantified as described in Supplementary Methods.

### 3. TRIP Pool Establishment, Aging, and NGS Library Preparation

All stable pools and cell lines generated here were derived from the parental CHO-K1 host cell line described previously. By Hilliard and Lee (Biotechnol. Bioeng. 118, 659-675 (2021)). Unless otherwise stated, cultures were grown at various scales using standardized culture conditions, working volumes, and shake speeds (Table 2). PiggyBac and lentiviral TRIP pools were established. Established TRIP pools were split into replicate cultures that were passaged independently for one week before sampling for next-generation sequencing (NGS) library preparations. TRIP pools were maintained in 125 mL shake flasks in the absence of selection pressure by passaging every three days to a seeding concentration of 0.4×10⁶ viable cells/mL to ensure a minimum of 10 million cells were carried forward. Additional samples were taken for library preparations from independently aged replicate flasks at milestones of 36 and 72 population doublings (PDLs) relative to the first extraction timepoint. RNA and genomic DNA were extracted from cells growing in mid-exponential phase on Day 2 of the first passage exceeding each PDL milestone. NGS libraries were prepared from RNA and genomic DNA extracted at each timepoint as described in Supplementary Methods to quantify mRNA expression and DNA copy number and map the integration site of each barcode.

### 4. TRIP NGS Data Preprocessing and Differential Expression Analysis.

Raw TRIP sequencing data was processed using a custom analysis pipeline. Forward reads from the expression and normalization libraries and reverse reads from the mapping libraries were trimmed with Cutadapt and counted for each barcode using custom R scripts. Forward reads in the mapping library were trimmed to exclude the end of the 3′ Piggybac or lentiviral terminal repeat used for primer binding and to eliminate chimeric sequences arising from ligation of multiple short DpnII/NlaIII fragments within the sequenced region. Trimmed reverse mapping reads shorter than 8 bp were discarded. The remaining mapping reads were mapped to the Chinese hamster PICRH genome assembly (CriGri-PICRH-1.0, GCF_003668045.3) with the Burrows-Wheeler Aligner and low-quality alignments (MAPQ<10) were filtered out of the dataset. Forward mapping reads with each TRIP barcode were re-paired with their reverse reads with associated mapping information (chromosome coordinate and strand) using a custom R script. Mapping tables for each timepoint were then merged into one summary table, keeping only the mapping with the highest read depth for each barcode. To account for minor offsets in alignment coordinates and mutations that may have occurred during culturing or library preparation PCRs, barcodes mapping to positions within 5 bp of each other were grouped and their normalization and expression read counts were added together, generating one consensus barcode and normalized expression value per integration.

Differential expression analysis was performed using a custom R script. Barcodes for which normalized expression could not be calculated due to read counts of zero in any of the normalization libraries were filtered out of the dataset. The barcodes were additionally filtered to eliminate barcodes with low read counts in either the expression or normalization libraries using the edgeR filterByExpr function Log₂ normalized expression values were calculated and adjusted to account for filtered library size differences between each replicate and timepoint. Cross-sample normalization by cyclic lowess normalization was then performed using the limma package to eliminate systematic bias between samples. Differential expression analysis was then performed with the limma package using a generalized linear model.

### 5. Generation and Evaluation of Master Host Cell Lines and RMCE Pools

Master host cell lines were generated by targeted integration of a landing pad at the Fer1l4 hotspot and eight Piggybac TRIP integration sites using the double-nick-donor CRISPR/HDR method (Table 1). CRISPR target sites within a 1,000 bp region surrounding each Piggybac integration site with high target specificity and editing efficiency scores were selected using the CRISPOR sgRNA design tool. The Chinese hamster PICR genome assembly (CriGri-PICR, GCF_003668045.1) was used as a background for specificity score calculations. Master host cell lines with single-copy integrations at each target site were generated by co-transfection of CHO-K1 host cells with a plasmid expressing an HDR donor-targeting sgRNA and hCas9_{D10A} and a landing pad plasmid modified with site-specific homology arms and a genomic locus-targeting sgRNA expression cassette in its backbone. Transfection-grade landing pad plasmids were prepared using a Plasmid Midiprep kit (Zymo Research) following the centrifugation protocol. CD4+ cells in the initial transfected pool were enriched on Day 8 post-transfection by one round of MACS with a Dynabeads® CD4 Positive Isolation kit (Thermo Fisher). Sorted CD4+ cells were recovered in a static 6-well plate and then scaled up to a 10 mL working volume in spin tubes over 6 days. Single-cell cloning was then performed by plating cells from the enriched pools in ClonaCell ACF semi-solid media (Stem Cell Technologies) in 96-well plates with a target of one cell per well. After twelve to fourteen days, clonally-derived colonies were picked manually, deposited into a 96-deep well plate, and shaken for four days.

On-target, single-copy integrations were verified by qualitative Junction PCR and droplet digital (ddPCR) copy number analysis using genomic DNA extracted at the 96-deep well plate stage. Taqman primer/probe assays (IDT) were designed to target the CD4 transgene, GFP-Puro^{r}, FCU1, Neo^{r}, and endogenous housekeeping genes RAB10 and HPRT1. RAB10 and HPRT1 were assigned values of two copies and one copy per diploid genome, respectively, based on NGS coverage-based copy number estimation reported previously for the parental CHO-K1 host cell line used here. (Hilliard and Lee, Biotechnol. Bioeng. 118, 659-675 (2021)). Genomic DNA samples were prepared for ddPCR analysis by a preliminary digestion step with HindIII-HF (New England Biolabs) and analyzed on a QXOne (BioRad) with ddPCR Supermix for Probes (no dUTP) following manufacturer instructions. Cell lines with the desired genotype were progressively scaled up and allowed to stabilize in spin tubes over two to three 3-day routine passages before banking and extracting RNA. mRNA expression analysis with ddPCR was performed using One-Step RT-ddPCR Master Mix following manufacturer instructions.

RMCE stable pools were generated by co-transfection of master host CHO-K1 cell lines with pCAG-NLS-HA-Bxb1, a gift from Pawel Pelczar (Addgene plasmid #51271), and a cargo plasmid. Transfected cells were passaged every three to four days under selection with 500 μg/mL Geneticin (Gibco) for sixteen days. RNA extractions from selected RMCE pools were performed using an RNA 96 Extraction kit (Zymo Research) with on-column DNase treatment. Top-performing stable pools for mAb expression were subjected to negative selection with 1 μM 5-fluorocytosine with the continued presence of Geneticin and passaged for another seven days at which point the pools had fully recovered. The selected pools were then passaged for an additional six days in fresh media without selection agents and scaled up to 125 mL shake flasks for fed-batch analysis.

### 6. Batch and Fed-Batch Analysis

The 4-day batch productivity screen was performed using Geneticin-selected mAb-expressing stable pools in 24-deep well plates. Batches were initiated at a seeding concentration of 0.5×10⁶/mL using cells split off from selecting pools after recovery from Geneticin selection and before addition of 5-fluorocytosine. Batches were performed in the absence of any selection agent. On Day 4, viable cell concentration was measured and cell culture supernatant was harvested by centrifugation for secreted mAb concentration quantification. Fed-batches were performed in 125 mL shake flasks with daily measurements of viable cell concentration and viability. Glucose and lactate concentrations in the cell culture supernatant were also measured daily using a YSI metabolite analyzer. Bolus additions of Cell Boost™ 7a and 7b (Cytiva) were performed starting on Day 3 following a pyramid feeding schedule (Table 3) and cultures were shifted to 32 °C on Day 4. Bolus additions of a supplemental pure glucose feed were also performed starting on Day 2 with a variable volume of addition to maintain excess glucose in the culture over 24 hours. Secreted mAb concentration in batch and fed-batch samples was quantified using ProA probes on a ForteBio Octet 96e following the manufacturer's protocol (Pall).

### B. Results

1. Position Effects on CMV Promoter-Driven Expression and Stability were Probed by Thousands of Reporters Integrated in Parallel

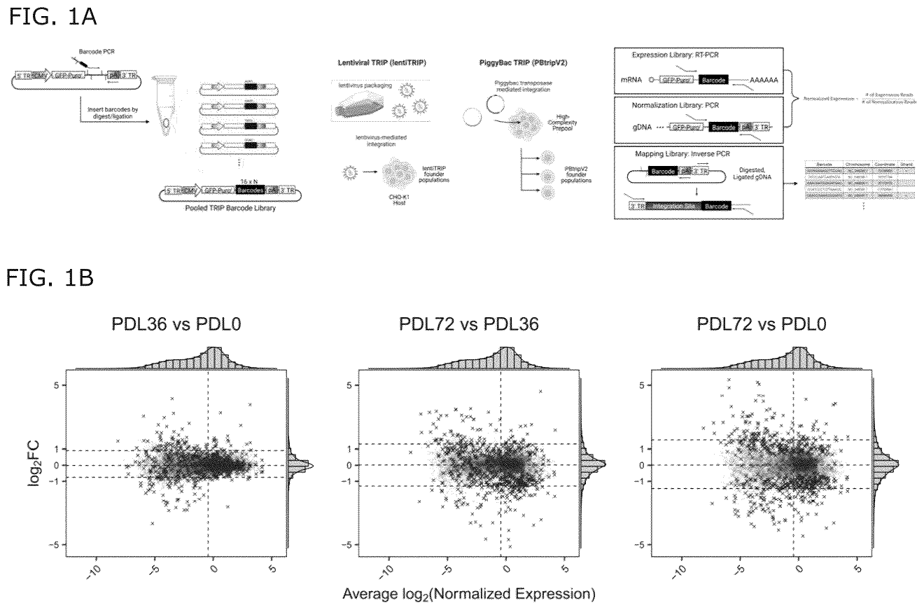

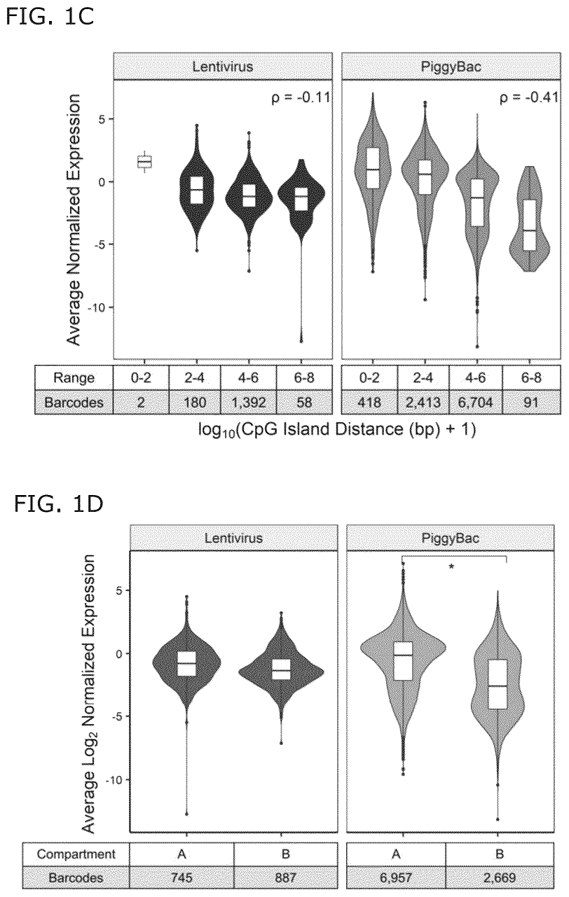

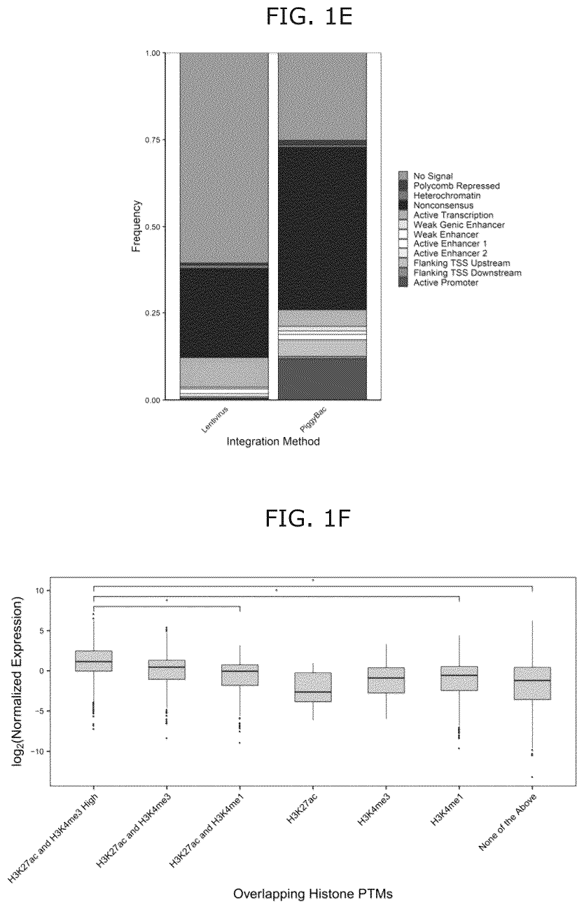

CMV-driven expression was measured at thousands of integration sites over 72 population doublings (PDLs) by adapting the TRIP pooled screening method for use in suspension CHO cells in both Piggybac and lentiviral formats (FIGS. 1A-C). The Piggybac dataset achieved a genome coverage 4.7- to 5.9-times greater than the lentivirus dataset. Differential expression analysis and precise integration site mapping could be performed for 9,663 Piggybac and 1,645 lentivirus barcodes using the first two aging time points at PDL0 and PDL36. However, only 6,322 Piggybac and 1,353 lentivirus barcodes were measurable at all three timepoints. The lower initial genome coverage in the lentivirus dataset is likely attributable to bottlenecking during lentivirus packaging, an inaccurate lentivirus infectious titer measurement, or variability in the transduction procedure. The absolute genome coverage of each pool would be challenging to estimate, as the final barcode count is the result of several filtering steps including integration site mappability and minimum expression cutoffs. However, Piggybac and lentiviral integration sites are expected to be subjected to similar biases during this filtering process. The final barcode count should therefore scale similarly with original genome coverage across methods. Age-dependent library quality deterioration was the result of competitive outgrowth, with fast-growing subpopulations in the cell pool outgrowing other subpopulations leading to low expression and normalization read counts for a significant fraction of barcodes in the PDL72 samples. This shift in barcode representation over time within each pool led to sequencing bias across timepoints, which was filtered out during integration site stability analysis by cross-sample normalization.

The majority of TRIP barcodes exhibited stable expression over time. Over 90% of Piggybac and lentivirus barcodes had an absolute log-fold change less than one between PDL0 and PDL36s. Additionally, only 627 out of 6,322 Piggybac barcodes and 5 out of 1,353 lentivirus barcodes were significantly differentially expressed (FDR<0.1) between any of the three culture ages. Differential expression analysis of PDL0 and PDL36 samples processed independently from the lower quality PDL72 data Identified 1,031 out of 9,663 Piggybac barcodes and 27 out of 1,645 lentivirus barcodes with differential expression (FDR<0.25). Elevated false discovery rates were used to minimize the risk of false negatives, as the elimination of potentially unstable sites was considered to be a higher priority than retaining every stable candidate site. Statistical power was also limited in this study by the use of biological duplicates during the aging process but the low frequency of differentially expressed barcodes obtained using either a log-fold change or an elevated false discovery rate cutoff indicated that the use of these integration methods in general led to stable transgene expression over time.

2. The Highest-Expressing Piggybac Integration Sites were Defined by a Limited Set of Genetic and Epigenetic Traits

Hotspot features defined based on DNA sequence motifs alone could provide a rough map of desirable integration sites for any cell type if it has an associated high-quality genome assembly. The highest-expressing Piggybac barcodes exclusively integrated within the boundaries of genomic CpG islands and a strong distance-dependent relationship was also observed in which the average expression of Piggybac barcodes decreased with increasing distance between the barcode integration site and the nearest CpG island (FIG. 1D). This relationship was not observed for the lentiviral integration sites, which exhibited consistent average expression across all distances, aside from the two barcodes that integrated within CpG islands.

The strength of association between integration site epigenetic properties and transgene expression was evaluated by comparing TRIP barcode integrations with published CHO epigenome data. (Hilliard and Lee (Biotechnol. Bioeng. 118, 659-675 (2021)). Piggybac barcode expression from integration sites in previously defined safe harbor regions in the CHO genome (Hilliard and Lee (Biotechnol. Bioeng. 118, 659-675 (2021)) was significantly higher than expression outside of these regions (Wilcoxon rank sum test, p=1.13×10⁻³⁴) but with a small effect size of 0.124, indicating that the combination of low-resolution 3D chromatin organization factors used to define these safe harbor regions was not effectively enriching for heightened transgene expression potential. A/B compartmentalization alone was a better predictor of expression (FIG. 1E). Piggybac barcodes with unstable expression between PDL0 and PDL36 (samples processed independently from PDL72, FDR<0.25) occurred at similarly low frequencies of 9.4% in safe harbor regions and 11.0% in the rest of the genome, indicating the combination 3D chromatin organization factors defined previously were also not prerequisites for stable expression in the practical context of a Piggybac transposon transgene expression cassette.

Histone post-translational modifications and CpG methylation were more effective predictors of high transgene expression than 3D chromatin organization factors. Piggybac- and lentivirus-mediated integration methods had distinct preferences for different histone modification combinations, with Piggybac barcodes primarily integrating in regions marked by H3K4me1, H3K4me3, and H3K27ac and lentivirus barcodes overlapping with H3K36me3 and H3K4me1 (Table 4). Elevated Piggybac barcode expression was also associated with above-average H3K27ac and H3K4me3 ChIP enrichment signals (FIG. 1F). In contrast, lentiviral integration sites marked with H3K4me1 or H3K36me3 did not have appreciable differences in expression relative to regions devoid of any histone modification ChIP signal enrichment (Wilcoxon rank sum tests, p=2.62×10⁻⁶-0.999, effect sizes=0.010-0.123). Piggybac and lentivirus barcodes also differed in their bias toward hypomethylated genomic regions (1 kb tiles with less than 10% CpG dinucleotide methylation), with integration frequencies of 17.6% (1,701/9,663) and 1.1% (18/1,645), respectively. Average Piggybac barcode expression in hypomethylated regions was significantly higher than in mid-level and hypermethylated regions, with moderate-to-large effect magnitudes (Wilcoxon rank sum tests, p=2.29×10⁻²²²-1.38×10⁻¹³⁶, effect size=0.312-0.532). In contrast, minimal differences in average expression between hypomethylated, mid-level, and hypermethylated lentiviral integration sites were observed (Wilcoxon rank sum tests, p=0.013-0.177, effect size=0.041-0.106), although these estimates were impacted by the presence of only 18 lentivirus barcodes in hypomethylated regions.

3. TRIP Integration Sites Retargeted for Landing Pad Integrations Exhibited Clonal Variability and Locus-Specific Interactions with Exogenous Regulatory Elements

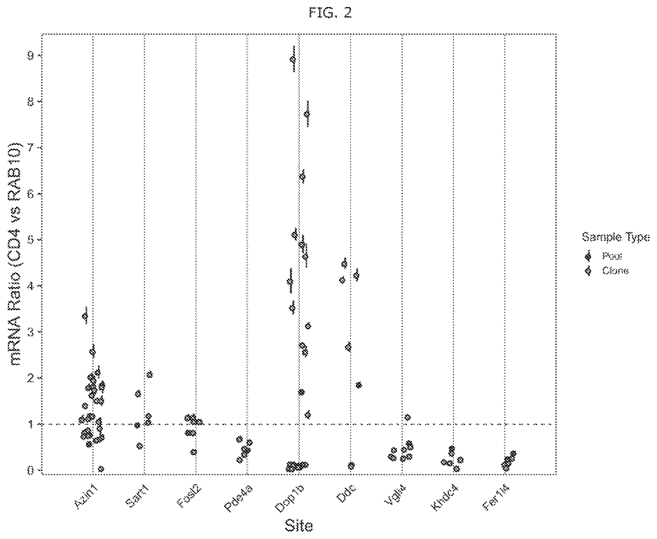

Eight Piggybac integration sites with median to maximum TRIP barcode mRNA expression and the Fer1l4 integration site used by Pfizer were retargeted for integration of a landing pad to evaluate the reproducibility of transgene expression from TRIP integration sites in a context directly translatable to a commercial CHO CLD platform. The eight TRIP sites were classified into “High” (Azin1, Sart1, Fosl2), “Mid” (Pde4a, Dop1b), and “Low” (Vgll4, Khdc4, Ddc) expression groups based solely on their TRIP barcode expression levels, with arbitrarily defined boundaries between groups. The genetic and epigenetic characteristics of this limited set of loci vary widely and are shared by other integration sites in all three expression groups. As Piggybac integration sites often do not directly overlap a suitable spacer and PAM sequence, Cas9 target sites in a range of 1000 bp surrounding the Piggybac insertion site were chosen based on computationally predicted high off-target stringency and on-target efficiency. All of the retargeted TRIP integration sites had equal or higher CMV-driven transgene mRNA expression than Fer1l4 in equivalent culture conditions (FIG. 2). Additionally, transgene mRNA expression from integration sites other than the hypervariable Dop1b and Ddc loci followed a similar trend to the TRIP predictions.

Master host cell lines with landing pad integrations at Dop1b and Ddc exhibited a high level of clonal variability in transgene mRNA expression relative to other sites. Approximately 50% of Dop1b cell lines had low transgene expression, consistently about 5-fold lower than the housekeeping gene, while the other 50% of Dop1b cell lines had higher expression than the housekeeping gene. Separation of Dop1b cell lines into high- and low-expression bins of approximately equal size was hypothesized to have been the result of allele-specific regulation of transgene expression and is a subject of continued investigation. Clone-specific chromosomal rearrangements surrounding the Dop1b locus could potentially also play a role in determining transcriptional activity in individual clones, given the inherent plasticity of the CHO genome. However, the consistency of the 50/50 split in expression bins across two cloning campaigns indicates that these rearrangements were not random or non-reproducible, if they occurred. Experimental factors such as potential sources of variability in culture conditions and cell health early in the single-cell cloning and outgrowth process could also have a long-term impact on the epigenetic characteristics of the locus. Additional experiments will be needed to determine if clone-specific regulation of transgene expression at the Dop1b locus can be associated with local genetic or epigenetic properties.

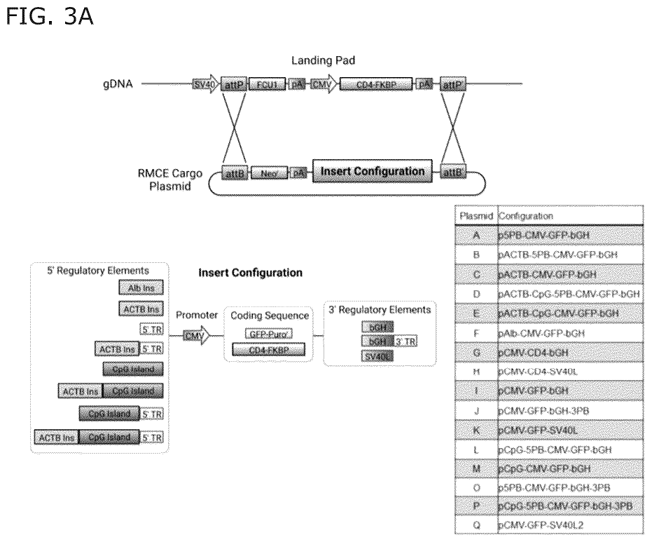

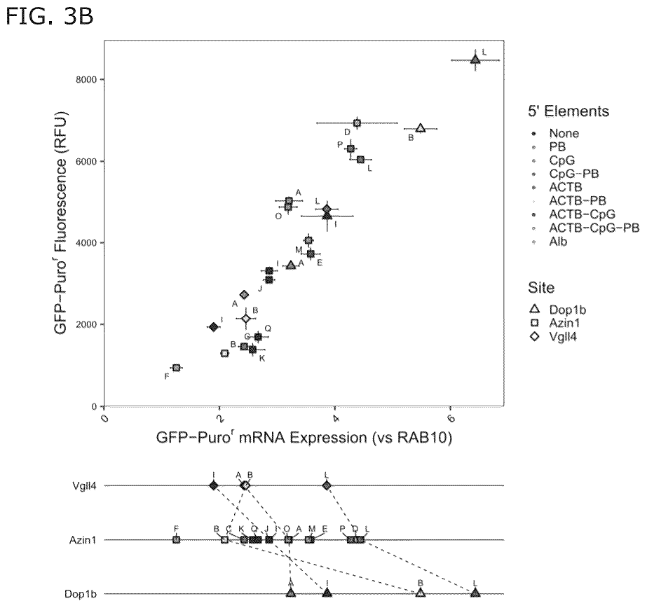

The potential impact of exogenous regulatory elements used in the Piggybac TRIP and landing pad systems on the translatability of the high-throughput screening results were probed by exchanging RMCE donor cassettes of varying configurations with the landing pad in a subset of master host cell lines with three different integration sites (FIG. 3A). Positioning the 5′ Piggybac ITR or a CpG island isolated from the Azin1 locus directly upstream of the CMV promoter in the RMCE donor cassette enhanced GFP-Puro^{r} expression relative to the CMV promoter alone following RMCE at the Azin1 integration site (FIG. 3B). Additionally, a synergistic enhancement effect was observed when these two elements were used in combination, with GFP-Puro^{r} expression achieving the highest observed level of any tested construct. Inclusion of the ACTB insulator element upstream of the CMV promoter led to the lowest GFP-Puro^{r} expression from the Azin1 integration site. The enhancement effect of the 5′ Piggybac ITR was also lost when the ACTB insulator was placed upstream of the ITR/CMV combination. However, this insulator had no effect on expression when it was positioned upstream of the CpG island. The CpG island/ITR/CMV combination universally enhanced expression and also led to the highest observed GFP-Puro^{r} expression levels at Dop1b and Vgll4. However, context-dependent effects were observed when using other combinations of the 5′ Piggybac ITR, ACTB insulator, and Azin1 CpG island at Dop1b and Vgll4, possibly reflecting interactions between the transgene expression cassette and endogenous regulatory elements arranged differently at each locus.

### 4. The Optimal mAb Production Plasmid Configuration was Product-Specific

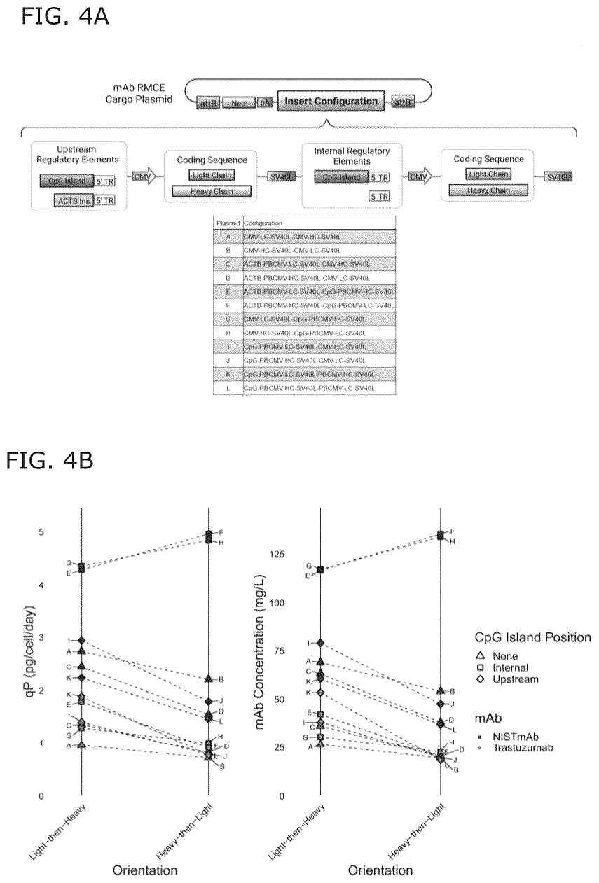

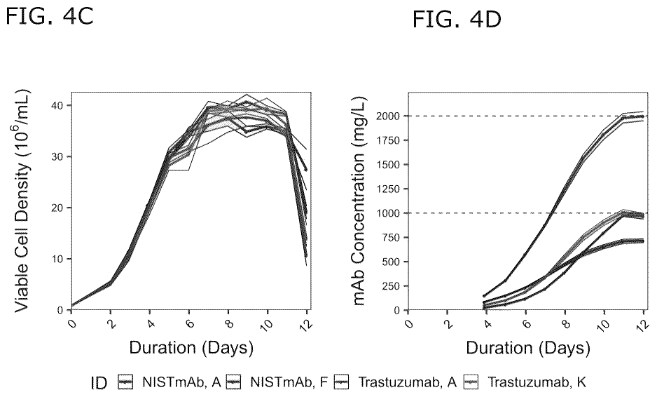

Monoclonal antibody productivity from the novel Dop1b hotspot was benchmarked using a master host cell line chosen based on high transgene mRNA expression and RMCE donor cassettes containing various combinations of 5′ regulatory elements found to enhance transgene expression in the initial GFP-Puro^{r} screen (FIG. 4A). A 3.7-fold (37 to 135 mg/L) and 2.9-fold (19 to 54 mg/L) range in configuration-dependent mAb productivity for NISTmAb and trastuzumab, respectively, was observed after a 4-day batch with stable pools post-RMCE (FIG. 4B). A direct comparison of the GFP-Puro^{r} pre-screen and mAb batch screen was not possible at configuration level due to the use of two transcription units for mAb expression. However, the demonstrated ability of many of the tested regulatory element combinations to increase cell-specific productivity relative to the “baseline” constructs (NA/NB for NISTmAb and TA/TB for trastuzumab) implied that GFP-Puro^{r} was an effective screening tool to identify regulatory elements with the potential to enhance mAb expression. Equivalent regulatory element combinations also led to different cell-specific productivity patterns between mAbs, with all novel trasutuzmab constructs leading to equivalent or enhanced cell-specific productivity relative to the baseline TA/TB constructs while NISTmAb productivity was enhanced or inhibited by the presence of the same elements in the same positions. These findings suggest that a pre-screen of genetic elements using a reference standard mAb rather than a fluorescent protein would likely have a similar success rate when applying findings to other mAbs. Despite the unpredictable differences observed between these products, this panel was able to capture enough variability in light chain and heavy chain expression to identify constructs with at least a two-fold improvement in both cell-specific and bulk productivity relative to the best “baseline” conditions that did not incorporate extra regulatory elements for both products (constructs NA/NB for NISTmAB or TA/TB for trastuzumab). In fed-batch mode, NISTmAb configuration F had the highest bulk productivity of any of the tested stable pools with a final mAb concentration of 1.996±0.012 g/L, a 2.8-fold improvement over NISTmAb construct A. Cell-specific NISTmAb productivity between fed-batch days 5 and 10 also increased from 2.93±0.06 pg/cell/day with construct A to 8.23±0.22 pg/cell/day with construct F. Trastuzumab constructs K and A exhibited a greater than 2-fold difference in mAb productivity in the first four days but reached similar final mAb concentrations of 0.987±0.012 g/L (Day 5-10 qP: 4.37±0.22 pg/cell/day) and 0.966±0.012 g/L (Day 5-10 qP: 4.63±0.11 pg/cell/day), respectively (FIGS. 4C-D). These changes in mAb productivity were not associated with any differences in cell growth or glucose metabolism. Bulk mAb productivities observed here were lower than other systems reported by industrial sources in the literature but achieved the same order of magnitude, indicating the Dop1b hotspot was capable of supporting production levels required for an industrial expression platform.

### C. Discussion

The high-throughput screen described here evaluated thousands of integration sites in the CHO genome for expression strength and stability over time. Over 90% of these integration sites maintained stable transgene expression levels over an extended culture aging timeline of 36 to 72 PDLs, in agreement with previous reports that transposase- and lentivirus-mediated integration methods in general lead to stable expression. The Piggybac system was a more effective screening tool for identifying stable hotspots than lentiviruses, which were insulated from both positive and negative position effects. Additionally, the integration bias of the Piggybac transposase toward active regulatory elements led to the identification of a set of loci in which the median mRNA expression level exceeded that of an industrial reference hotspot known to be capable of supporting multi-gram per liter volumetric mAb productivity. One of these hotspots supported mAb productivities of approximately 0.7 to 2 g/L in fed-batch benchmarking experiments, which could be increased further by optimizing culture conditions or using a host cell line with a more optimal balance between growth and protein production. Generally, to confirm long-term stability of a particular transgene into newly identified hotspots resulting in increased titers, one would still need to perform dedicated analyses of each selected clone. It may also be of interest to compare the performance of a Dop1b targeted integration platform against a reference random integration or Piggybac CLD platform in the future.

Continued evaluation of highly ranked sites from the Piggybac TRIP dataset in a landing pad context could eventually validate thousands of additional hotspots suitable for biomanufacturing applications. Transgene mRNA expression in stable polyclonal pools generated in this study using the double-nick-donor CRISPR method was representative of the average transgene mRNA expression in clones with the same integration site. Therefore, stable pools could be used to perform a preliminary validation of a limited number of sites from this dataset in as little as two weeks. In cases where CRISPR methods are not accessible due to licensing challenges, alternative targeted integration methods (e.g., ZFNs) may be used to target a limited number of sites. Different research groups could select panels of potential hotspots for evaluation either directly by rank in the TRIP dataset or by using additional cell line-specific ChIP-seq (H3K27ac and H3K4me3) and bisulfite sequencing profiles to account for differences in cell lines and culture conditions between labs.

Genome-wide transgene expression patterns obtained using the TRIP system suggest that Piggybac transposons are more sensitive to the position effect than lentiviruses and that each integration method could be preventing epigenetic silencing of its cargo using a different mechanism. Elevated Piggybac integration frequencies in endogenous CpG islands with active histone modifications and hypomethylated CpG dinucleotides indicated that negative position effects were avoided in the Piggybac transposase system by targeting sites within or near constitutively active regulatory elements. Plasmid configuration experiments described here also support a model in which the active expression of transgenes within engineered Piggybac transposons is stably maintained by interactions with the nearest upstream active genomic regulatory element mediated by the 5′ Piggybac ITR. In contrast, lentiviruses were not biased toward canonical epigenetic markers of active regulatory elements, including CpG hypomethylation, and could be avoiding the position effect through some other method, possibly by viral regulatory elements within the lentivirus package acting as chromatin domain insulators. Alternatively, the absence of position-dependent effects on transgene expression from lentivirus integrations could be explained by a lack of variety in the epigenetic environments surrounding the surveyed lentiviral integration sites. Regardless of how lentiviruses achieve position-independent expression, these results imply that the Piggybac transposase integration system is a more appropriate method than lentiviruses to use when searching for the highest-expressing hotspots with reproducible expression upon retargeting.

The distance-dependent relationship between Piggybac barcodes and CpG islands in such a small range indicates that the size of a hotspot region capable of maximizing transgene expression is on the order of 1 to 10 kb. Defining hotspots using genomic CpG islands alone limits the total search space to specific regions accounting for less than 3% of the Chinese hamster genome, which could be filtered further using H3K27ac/H3K4me3 histone modifications and CpG hypomethylation profiles, the best epigenetic predictors of high transgene expression out of any of the predictors evaluated here. Weak associations between low-resolution 3-dimensional chromatin structural signatures measured previously and TRIP barcode expression also indicated that more complex assays and analyses are generally not required to identify genomic regions with enhanced stability or hotspot potential. The flexibility of targeted integration methods allows for integration of a transgene into or near active regulatory regions of the genome, thereby eliminating the reliance on long-distance interactions mediated by 3-dimensional organization of chromatin at the previously measured resolution of 100 kb to enhance expression. However, context-dependent plasmid configuration results also support a model in which specific higher-resolution interactions between the promoter driving transgene expression and nearby endogenous regulatory elements play an important role in determining transgene expression level. Future experiments directly measuring perturbations in high-resolution chromatin looping between the transgene promoter and nearby endogenous regulatory elements caused by chromatin-modifying elements in the transgene expression cassette could provide additional evidence as to how transgene expression is regulated at individual hotspots by these mechanisms.

Consistent observations of clonal variability in transgene mRNA expression in cell lines with the same integration site highlighted a significant shortcoming of the TRIP system. Each expression measurement within the TRIP pool was obtained from a cell subpopulation grown out from a single integration event due to the intrinsic constraints of the DNA barcoding method. Therefore, highly ranked TRIP sites may only represent loci with the potential to support the generation of a cell line with desirable mRNA expression levels, rather than loci that would consistently achieve higher expression than any other actively transcribed location in the genome. An evaluation of clonal variability at many different integration sites using repeated TRIP experiments would be challenging, as the likelihood of repeated targeting of exact integration sites with the same orientation is low without increasing the complexity of the barcode library to achieve greater genome coverage per experiment. In the future, alternative high-throughput screen designs that implicitly incorporate clonal variability at each integration site within a pool could more effectively identify hotspots for which clones with maximized transgene expression can be isolated at high frequencies. These alternative methods would likely require the use of other novel targeted integration systems, such as CRISPR prime editing paired with recombinase-mediated insertion, to circumvent the technical limitations of barcoding clonal subpopulations by random integration and the throughput limitations of an HDR-mediated integration approach.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

TABLE 1
Retargeted integration site mapping information.

| | TRIP PICRH | | CRISPR PICRH | | SEQ ID |
|---|---|---|---|---|---|
| Locus | Coordinate | Strand | Coordinate | Spacer Sequence | NO |
| Azin1 | NC_048603.1: | + | NC_048603.1: | (g)tacggtgtgacgtgcaccgg | 1 |
| | 13,239,247 | | 13,249,147 | | |
| Sart1 | NC_048596.1: | − | NC_048596.1: | (g)gggagtcgaggatttgagta | 2 |
| | 53,947,823 | | 53,947,833 | | |
| Fosl2 | NC_048600.1: | + | NC_048600.1: | (g)aagttcagggcacgcgtggc | 3 |
| | 12,339,811 | | 12,339,906 | | |
| Pde4a | NC_048597.1: | + | NC_048597.1: | (g)agcagggccccgttagacta | 4 |
| | 134,028,574 | | 134,028,625 | | |
| Dop1b | NC_048597.1: | + | NC_048597.1: | (g)atcaccgcttgtccccctca | 5 |
| | 4,073,164 | | 4,073,448 | | |
| Ddc | NW_023276807.1: | + | NW_023276807.1: | (g)tctgcatctggctaaatggg | 6 |
| | 93,746,581 | | 93,746,684 | | |
| Vgll4 | NC_048601.1: | + | NC_048601.1: | (g)gagtcctcgaggccctagca | 7 |
| | 29,977,155 | | 29,977,002 | | |
| Khdc4 | NW_023276806.1: | − | NW_023276806.1: | (g)ctaatgagccaaccgggcag | 8 |
| | 65,852,036 | | 65,852,011 | | |
| Fer1l4 | N/A | + | NC_048599.1: | (g)ttcaagggctcagtgaccgt | 9 |
| | | | 23,557,651 | | |

TABLE 2
CHO culture conditions.

| | Working | Shake |
|---|---|---|
| Culture Vessel | Volume (mL) | Speed (rpm) |
| 96-deep well plate (square well) | 0.5 | 300 |
| 24-deep well plate (square well) | 2 | 200 |
| 24-well plate | 1 | 200 |
| 6-well plate | 2 | 135 |
| 50 mL spin tube | 10 | 200 |
| 125 mL Erlenmeyer flask | 25 | 135 |
| 500 mL Erlenmeyer flask | 100 | 135 |
| 1 L Erlenmeyer flask | 200 | 135 |

TABLE 3
NISTmAb and trastuzumab fed-batch feeding schedule.

| | Cell Boost | Cell Boost | Glucose |
|---|---|---|---|
| Day | 7a (% vol.) | 7b (% vol.) | Target (g/L) |
| 3 | 3 | 0.3 | 6 |
| 4 | 3 | 0.3 | 6 |
| 5 | 3 | 0.3 | 6 |
| 6 | 4 | 0.4 | 9 |
| 7 | 4 | 0.4 | 9 |
| 8 | 5 | 0.5 | 9 |
| 9 | 5 | 0.5 | 9 |
| 10 | 4 | 0.4 | 9 |
| 11 | 4 | 0.4 | 9 |

TABLE 4
Integration counts and frequencies of Piggybac (PB) and lentivirus
(LV) barcodes overlapping specific histone modifications.

| PTM | PB | PB % | LV | LV % |
|---|---|---|---|---|
| H3K27ac | 3274 | 33.9 | 54 | 3.3 |
| H3K27me3 | 38 | 0.4 | 1 | 0.1 |
| H3K36me3 | 1084 | 11.2 | 280 | 17.0 |
| H3K4me1 | 4303 | 44.5 | 260 | 15.8 |
| H3K4me3 | 3447 | 35.7 | 43 | 2.6 |
| H3K9me3 | 43 | 0.4 | 9 | 0.5 |
| No Signal | 3239 | 33.5 | 1141 | 69.4 |

Histone modification ChIP-seq profiles used for overlap analysis were originally collected by Feichtinger et al. (Biotechnol. Bioeng. 113, 2241-2253 (2016)) and reprocessed as described previously by Hilliard and Lee (Biotechnol. Bioeng. 118, 659-675 (2021)).

1. A recombinant Chinese Hamster Ovary (CHO) cell for producing a recombinant protein, comprising a heterologous gene encoding the recombinant protein, wherein the heterologous gene is integrated into the genome of the recombinant CHO cell at one or more integration sites, wherein each of the one or more integration sites is within 200 kb of a target sequence in the genome, wherein the target sequence is at least 80% identical to a gene sequence selected from the group consisting of Azin1, Sart1, Fosl2, Pde4a, Dop1b, Vgll4, Khdc4 and Ddc.

2. The recombinant CHO cell of claim 1, wherein the heterologous gene is integrated into the genome of the recombinant CHO cell at the one or more integration sites in the one or more target sequences.

3. The recombinant CHO cell of claim 1, wherein the heterologous gene is integrated into the genome of the recombinant CHO cell at the one or more integration sites within 200 kb of one of the one or more target sequences.

4. The recombinant CHO cell of claim 1, wherein the heterologous gene is integrated into the genome of the recombinant CHO cell at the one or more integration sites in one of the one or more target sequences.

5. The recombinant CHO cell of claim 1, wherein the heterologous gene is integrated into the genome of the recombinant CHO cell at one of the one or more integration sites in one of the one or more target sequences.

6. The recombinant CHO cell of claim 1, wherein the recombinant CHO cell is incubated in a culture medium and expresses the recombinant protein.

7. The recombinant CHO cell of claim 1, wherein the recombinant protein is a monoclonal antibody.

8. A method for producing a recombinant protein, comprising incubating the recombinant CHO cell of claim 1 in a culture medium, and expressing the recombinant protein by the recombinant CHO cell.

9. The method of claim 8, further comprising producing the recombinant protein at 0.1-10 g/L based on the total volume of the culture medium or 1-10 pg per said recombinant CHO cell per day.

10. The method of claim 8, further comprising purifying the recombinant protein from the culture medium.

11. The method of claim 8, wherein the recombinant protein is a monoclonal antibody.

12. A method for preparing a recombinant CHO cell, comprising obtaining a host CHO cell comprising a genome having one or more target sequences, wherein each of the one or more target sequences is at least 80% identical to a gene sequence selected from the group consisting of Azin1, Sart1, Fosl2, Pde4a, Dop1b, Vgll4, Khdc4 and Ddc; and integrating a heterologous gene encoding a recombinant protein into the genome at one or more integration sites within 200 kb of the one or more target sequences, whereby a recombinant CHO cell is prepared.

13. The method of claim 12, further comprising integrating the heterologous gene into the genome at the one or more integration sites in the one or more target sequences.

14. The method of claim 12, further comprising integrating the heterologous gene into the genome at the one or more integration sites within 200 kb of one of the one or more target sequences.

15. The method of claim 14, further comprising integrating the heterologous gene into the genome at the one or more integration sites in one of the one or more target sequences.

16. The method of claim 12, further comprising integrating the heterologous gene into the genome at one of the one or more integration sites in one of the one or more target sequences.

17. The method of claim 12, wherein the genome comprises a landing pad.

18. The method of claim 12, further comprising incubating the recombinant CHO cell in a culture medium, and expressing the recombinant protein.

19. The method of claim 18, further comprising purifying the recombinant protein from the culture medium.

20. The method of claim 12, wherein the recombinant protein is a monoclonal antibody.
